(19) United States
(12) Patent Application Publication
(10) Pub. No.: US 2007/0193590 A1
(43) Pub. Date: Aug. 23, 2007

(54) Method Of Determining Nervous System Malfunctions

(76) Inventor: Michael A. Lorente, Clawson, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 10/575,638

(22) PCT Filed: Oct. 13, 2004
(86) PCT No.: PCT/US04/33723

§ 371 Date: Feb. 27, 2007

Related U.S. Application Data
(60) Provisional application No. 60/511,028, filed on Oct. 13, 2003.

(51) Int. Cl. A61B 19/00

(52) U.S. Cl. 128/898

(57) ABSTRACT

A method for clinically determining nervous system malfunction by exerting pressure at specific pressure points on the patient's body and measuring the changes in the patient's leg length in response to the pressure being applied at the pressure points.

### RELATED APPLICATION

This application claims priority of U.S. Provisional Patent Application Ser. No. 60/511,028 filed Oct. 13, 2003, which is incorporated herein by reference.

### FIELD OF THE INVENTION

The present invention relates to natural healthcare methods for clinically determining nervous system malfunctions and more particularly to a dynamic method for determining nervous systems malfunctions caused by spinal pressure on a nerve branch.

### BACKGROUND OF THE INVENTION

It is appreciated and well understood by healthcare professionals that only nerves feel pain. Neither skin, bones, muscles, nor anything else feels pain unless a nerve is connected to it. All nerves come from the brain, go down through the spine and communicate life-giving messages out to every living cell in the human body. If a message is altered because of a pinched nerve, then the body falls to a lower state of health and healing which usually results in a person experiencing weakness, pain and, in some cases, disease that stems from the person experiencing chronic nerve pressure.

Traditionally, static methods have been used to evaluate nervous system malfunctions associated with nerve pressure being experienced at various points within the human body. These methods typically require medical health professionals to obtain x-ray photos of a patient and thereafter to review the x-ray photos to determine locations within the body where nerve pressure may be present. However, this x-ray method of static evaluation can sometimes require that the patient be subjected to this procedure many times before the nervous system malfunction can be properly analyzed or treated. Such a result subjects the patient to numerous photos of potentially damaging x-ray exposures and yet with this method only a static evaluation is possible.

### SUMARY OF THE PRESENT INVENTION

Advantageously, this present invention provides a pioneering method of determining a nervous system malfunction in a dynamic fashion by providing a reliable and sufficient indication of what nervous system malfunctions exist within a person such that the need for x-ray evaluations can be substantially reduced and/or minimized. Additionally, this new dynamic analysis checks the living, breathing, functional body responses of the patient to provide correct and updated information on the patient's changing condition as desired.

### DESCRIPTION OF THE DRAWINGS

A better understanding of the present invention can be had by referring to the following description and to the drawings in which:

FIG. 1 is a Merric Chart and including an anatomical representation of a patient to illustrate the location of the pressure points utilized in the method of the present invention;

FIG. 2 is a view illustrating the method of the present invention being practiced on a patient lying on his back; and

FIG. 3 is a view illustrating the position of a patient's legs during practice of the horizontal check of the method of the present invention.

### DESCRIPTION OF THE INVENTION

The present invention provides a method of dynamically determining nervous system malfunction caused by spinal pressure on a nerve branch wherein the inventive method provides advantage over traditional static methods of determining nervous system malfunctions.

The method comprises carrying out a series of clinical observations of leg length changes as pressure is applied at four points along the pant leg meridian of a patient. Any resulting leg length change as pressure is applied to the predetermined points is noted by a healthcare professional performing the evaluation. The total leg length change indicates the severity of the problem by showing greater change in the length of the subject leg corresponding to a more severe problem, and the rate of change indicating how long the problem has existed.

Pressure point locations that cause leg length changes to occur will also indicate what particular part of the nervous system and/or area may be most affected. Although anyone can notice the leg length changes and notice a problem exists, a qualified healthcare professional is able to determine that malfunctions are likely in the body parts controlled by the nervous system branch affected at the location of the respective pressure points which are evaluated during the initial series of steps.

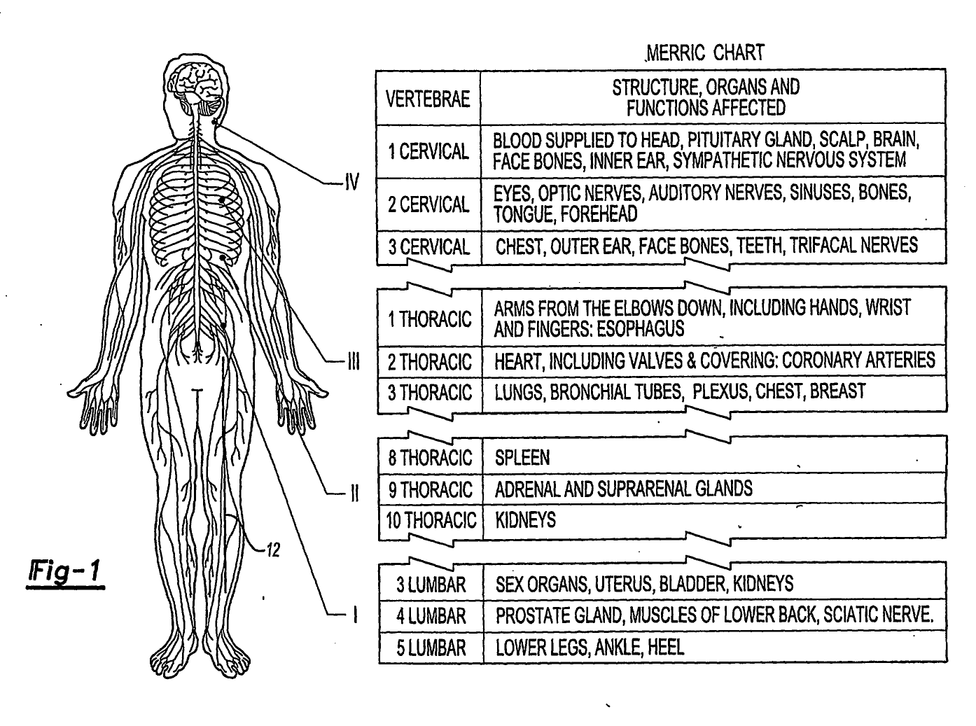

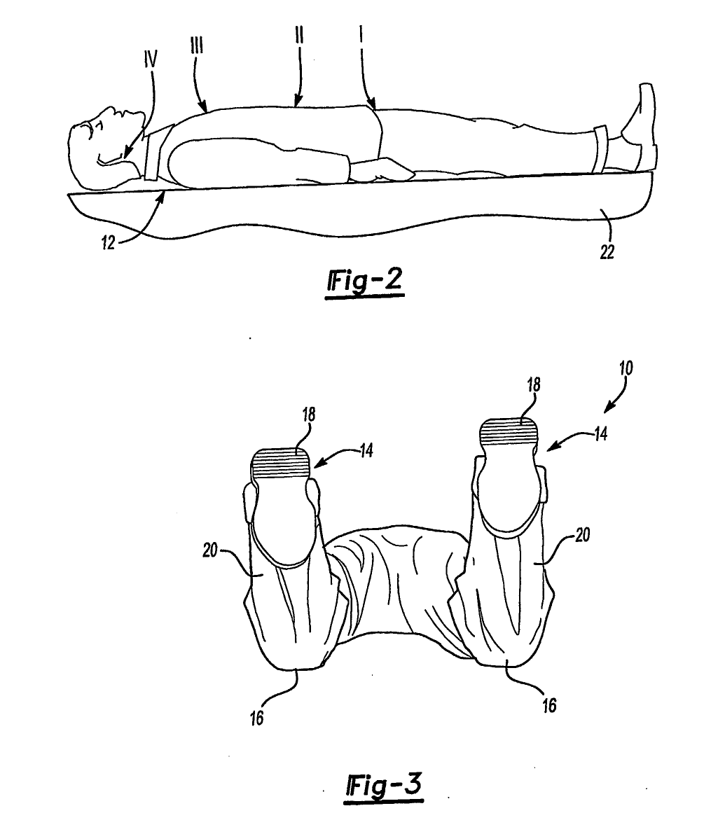

FIGS. 1 and 2 illustrate the vertical spine check steps associated with the method of the present invention for determining nerve pressure locations. Still referring to FIG. 1, there is a general anatomical illustration of a patient 10 along with a chart known as the Merric Chart illustrating various pressure points I-IV on the patient 10 for assisting the healthcare professional during patient evaluation according to the present invention. Accordingly, the healthcare professional may reference this illustration when performing the vertical spine check.

The vertical spine check is comprised of a first step of having the patient 10 (FIG. 2) lay down on their back whereby the healthcare professional can evaluate and determine the length of each of the patient's legs and thereafter establish a base difference between the legs. Once the base difference and leg length has been established, the healthcare professional continues the evaluation on the short leg side, or dominant hand side if both legs are even, to find a spot between the hip and navel which references nerves associated with various parts and organs as according to pressure point (I) as illustrated in the table and diagram of FIG. 1 based on the Merric and other proven body system experimentation. Once a proper spot is located, the healthcare professional presses lightly at that point making contact and thereafter notes the change in the patient's leg length in response to that pressure point application. The change in length is recorded for purposes to be described hereinafter.

A next step requires the healthcare professional to follow up the pant leg meridian 12 (FIG. 1) on the same side of the patient 10 to just under the rib cage to pressure point II as illustrated on the anatomical diagram of FIG. 1. This pressure point check is done and the leg length change from most recent position is noted and recorded.

The pressure points III and IV are located and evaluated in a fashion similar to pressure points I and II described above. Thereafter, the healthcare professional sums all the recorded changes in the patient's leg length to determine quantitative (the extent of the change) and qualitative (the rate of the change) to determine the extent of nervous system malfunction, if any, existing in the patient. By adding the changes in leg length recorded during the total evaluation, the healthcare professional is provided with a strong indication as to the severity of the patient's condition. Negative measurements or leg shortening may indicate spinal curvatures while the rate of change in the leg length is indicative of the age of the problems that the patient is experiencing.

The method that has been described so far is referred to the vertical spine check even though the patient 10 is lying down as shown in FIG. 2 while the measurements are being taken. The check is taken at vertically spaced pressure points I-IV along the spine of the patient 10.

The next series of steps in the inventive method of evaluating nerve pressure locations is referred to as the horizontal spine check (see FIG. 3). The horizontal spine check comprises the steps of first having the patient 10 lie down in the prone position. Thereafter, the patient's feet 14 are lifted freely by bending the knees 16 while the patient 10 is lying on her stomach on the examination table 22 (FIG. 2). The healthcare professional compares the difference in the height of the patient's heels 18 as the legs 20 reach a 90-degree angle with the table. The difference is noted and recorded by the healthcare professional.

Next, the healthcare professional adds the number obtained from the horizontal spine check to the total obtained from the vertical spine check to total and complete the method of nerve pressure evaluation in the patient. Again these totals tell the healthcare professional where spinal corrections need to be made.

The above dynamic evaluations are performed before corrections are made to the patient to reveal where the most significant areas are that changes need to be made in response to pressure contacts being applied to the pressure points as referenced by (I-IV) in FIG. 1. Accordingly, the healthcare professional is prompted to begin making corrections to the patient 10 at the locations where the most significant changes were noted first. After making the appropriate corrections, the healthcare professional can recheck the patient 10 to determine if the corrections were done properly and if the nervous system malfunction has been corrected. If the corrections have been made properly the patient's legs 20 will be of even length and there will be no change of length caused by applying pressure at the pressure points I-IV.

The foregoing description provides a dynamic method of evaluating nervous system malfunctions in a patient and provides a method desirous over traditional static methods of determining the same. By utilizing the inventive method herein, benefits are realized by both the healthcare professional and the patient whereby the time and cost of providing and receiving adequate care and more effective treatment for nervous system malfunctions is effected by performing the above-described steps.

Although I have described a preferred method, it is apparent to one skilled in the art that changes can be made to the described method without departing from the spirit of the invention as set forth in the attached claims.

I claim:

1. A method for determining a nervous system malfunction caused by spinal pressure in a nerve branch of a patient, said method comprising the steps of:
performing a four-point vertical spine check;
recording leg length change and rate of change information of the patient that occur in response to performing the four-point vertical spine check;
adding the leg length changes to obtain a total;
performing a horizontal spine check;
recording a difference in leg length noted from performing the horizontal spine check; and
adding the total obtained from the vertical spine check to the total obtained from the horizontal spine check to determine an indicator value wherein said indicator value and said rate of change information are used to assist a healthcare professional in determining the nervous system malfunction and to provide the healthcare professional with treatment information for analyzing and correcting the nervous system malfunction.

2. The method of claim 1 wherein the horizontal spine check comprises the steps of:
placing a patient in a prone position on an examination table;
lifting the patient's heels to a 90-degree angle with respect to the knees of the patient while maintaining the knees of the patient in contact with the examination table; and
noting and recording the difference in length of the patient's legs as the legs stand at the 90-degree angle.

3. The method of determining and treating a nervous system malfunction caused by spinal pressure in one or more nerve branches of a patient as defined in claim 1 and in which said vertical spine check is performed by applying pressure to the patient at four selected locations along an extension of the patient's pant leg meridian.

4. The method of dynamically determining and augmenting treatment of a nervous system malfunction as defined in claim 1 and in which said horizontal spine check comprises measuring and comparing the distance between each of the patient's legs and the examination table while the patient is in a prone position with the legs bent 90 degrees at the knees.
